(19) United States
(12) Patent Application Publication
(10) Pub. No.: US 2025/0338874 A1
(43) Pub. Date: Nov. 6, 2025

(54) Weaning Supplement Composition for Controlling Diarrhoea and Improving Performance of Weaning Animals

(71) Applicant: AVT NATURAL PRODUCTS LTD., Chennai (IN)

(72) Inventors: Ravichandran Sripathy, Chennai (IN); Kaliyarmattom Pushpakumari Narayanan, Chennai (IN); Emma Wall, Chennai (IN); Arunkumar Thudanal Chandrashekharan Nair, Chennai (IN); Vinay Kumar Suthapalli, Chennai (IN); Padmaja Rani, Chennai (IN)

(21) Appl. No.: 18/859,242

(22) PCT Filed: Apr. 25, 2023
(86) PCT No.: PCT/IN2023/050406

§ 371 Date: Oct. 23, 2024

(30) Foreign Application Priority Data
Apr. 25, 2022 (IN) 202241024167

(51) Int. Cl. A23K 10/30; A23K 20/158; A23K 20/163; A23K 40/30; A61K 36/61; A61K 36/81; A61K 36/8962

(52) U.S. Cl. CPC A23K 10/30; A23K 20/158; A23K 20/163; A23K 40/30; A61K 36/61; A61K 36/81; A61K 36/8962

(57) ABSTRACT

The present invention discloses a fat encapsulated and/or water-soluble composition (weaning supplement composition), comprising standardized capsicum oleoresin, garlic and clove essential oils, that can be used as a supplement or additive in livestock, to help prevent diarrhoea, mortality, secondary infections 10 and performance loss, in weaning animals.

### TECHNICAL FIELD OF THE INVENTION

The present invention relates to weaning supplement composition comprising standardized botanicals of essential oils and oleoresins useful in animal industries. More particularly, the invention relates to weaning supplement composition comprising capsicum oleoresin; clove oil, garlic oil and optionally vegetable oil encapsulated in fat soluble carrier or water-soluble carrier, which is pellet-stable, and is 100% dust free and a process thereof, to improve gut health, reduce diarrhoea incidences and improve performance of the weaning animals.

### BACKGROUND AND PRIOR ART

In normal circumstances, natural weaning is a slow process, where gradual decrease in milk supply from the mother and concomitant increase in solid food were done simultaneously and eventually efforts are taken to gradually reduce maternal-filial bond. In modern livestock production practices, weaning is done abruptly and much earlier compared to natural weaning of the species, this in turn exposes the weaning animals to range of physiological, social and environmental stressors¹. The stress of weaning alone causes inflammation in the gut and on top of that, weaning animals are faced with the stressors of being physically removed from lactating mother, introduced to new pen mates, and exposed to significant dietary changes all while genetics are pushing nutrient partitioning towards growth. Post-weaning diarrhoea has been a major challenge in the production of lactating animals, especially swine and ruminants, for years. The major causative agent for most post-weaning diarrhoea is enterotoxigenic E. coli and the symptoms include dehydration, death (up to 30% mortality), and decreased growth in infected animals². The economic implications of post-weaning diarrhoea are substantial due to the high mortalities and the long-term impact of disease on surviving animals. Prevention techniques are focused on animal management in the form of optimized passive immunity, decreased stocking density, high biosecurity, and optimized nutrition programs. In addition, botanical feed supplements with known intestinal effects represent potential solutions for both prevention of disease and increasing the animal's ability to cope in the case of an outbreak.

U.S. Pat. No. 9,241,502 B2 describes a composition containing encapsulated capsicum extract in combination with other ingredients including clay, that can be used as a supplement or additive in feed to reduce incidence and to treat symptoms of fescue toxicosis in mammals.

Another patent (U.S. Pat. No. 10,918,118B2) discloses a method of inhibiting diarrheal pathogens, porcine epidemic diarrhea virus (PEDv) and Salmonella bacteria, by supplementing chemical mitigant comprising a medium chain fatty acid, an essential oil, and/or sodium bisulfate. The chemical mitigant can be introduced to the feed or feed ingredients at an inclusion rate, from about 0.01 weight % to about 5 weight %. The essential oil may be selected from capsicum oleoresin, garlic oleoresin, turmeric oleoresin, rosemary extract, and or wild oregano essential oils. Both these patents detail the method of application of different botanicals in controlling diarrhea and/or diarrheal pathogens, however, in both these patents, enhanced livestock performance and improvements in host, such as improvement in intestinal integrity and gut health, were not observed.

R. Došen et al reports that the occurrence of disease of respiratory tract of swine, bacterial infections are mostly consequence of secondary infections, due to Pasteurella sp., Streptococci sp., Staphylococcus sp. and E. coli. The pneumoniae relatively rarely, caused by infection with Salmonella sp. and K. pneumoniae (Biotechnology in Animal Husbandry January 2007 DOI: 10.2298/BAH0702237D).

Although the above literature reports reducing diarrhea in animals and improving weight gain; still it is silent on decreasing respiratory diseases in swine caused due to E. Coli. Moreover, the literature fails to provide a weaning supplement composition that will improve intestinal health, function, and structure in swine.

Y. Liu et al 3 studied the effects of three different plant extracts (capsicum oleoresin, garlic botanical and turmeric oleoresin) on diarrhea, immune response, intestinal morphology, and growth performance of weaned pigs experimentally infected with a pathogenic F-18 Escherichia coli and concluded that the all the 3 plant extracts tested reduced diarrhea and inflammation caused by E. coli infection, however supplementation of these botanicals did not affect the growth performance of the pigs.

Based on these literatures it is clear that, there is a need for weaning supplement composition that controls diarrhea and improves performance of the weaning animals.

### OBJECTIVE OF THE INVENTION

Accordingly, it is the objective of the present invention to provide a weaning supplement composition that can reduce diarrhea incidence and also improves gut health, performance and general health of weaning animals and human beings.

Another objective of the present invention is to provide a process for preparing the weaning supplement composition.

### SUMMARY OF THE INVENTION

The present invention provides a weaning supplement composition that improves intestinal health, function, and structure in weaning animals and human beings. The composition can positively impact host by modulating gut microbiome, immunity and inflammatory responses of the host. The composition of the present invention decreases susceptibility to post-weaning diarrhea, prevent infection/improves disease resistance as well as potential downstream illnesses such as gut and respiratory disease in animals and also improves Performance in Weaning animals and human beings.

Accordingly, in one aspect, the invention provides a weaning supplement composition which comprises;
a) Capsicum oleoresin in an amount of 0.1 to 10%;
b) Clove oil in an amount of 0.1 to 20%;
c) Garlic oil in an amount of 0.1 to 20%;
d) Emulsifiers or Vegetable oil(s) in an amount of 0 to 30%; and
e) A water soluble or fat soluble carrier in an amount of 50 to 99%,

In another aspect, the invention provides composition of water-soluble weaning supplement in liquid form which comprises;
a) Capsicum oleoresin in an amount of 0.1 to 10%;
b) Clove oil in an amount of 0.1 to 20%;
c) Garlic oil in an amount of 0.1 to 20%;
d) Emulsifiers in an amount of 0 to 30% and
e) Water soluble carrier in an amount of 50 to 99%.

In yet another aspect, the invention provides a fat encapsulated weaning supplement composition which comprises;
a) Capsicum oleoresin in an amount of 0.1 to 10%;
b) Clove oil in an amount of 0.1 to 20%;
c) Garlic oil in an amount of 0.1 to 20%;
d) Vegetable oil(s) in an amount of 0 to 30%; and
e) Fat soluble carrier in an amount of 50 to 99%,

In another aspect, the invention provides a process for preparation of weaning supplement composition by method of spray encapsulation.

### DESCRIPTION OF DRAWINGS

FIG. 1 depicts process flow chart for the preparation of weaning supplement composition using fat soluble carrier according to the examples 1 and 2

FIG. 2 depicts the reduced diarrheal frequency in the piglets challenged with E. coli, when compared to Controlled piglets challenged with E. coli having a high diarrheal frequency. The four treatment groups were considered during the trial. Negative control is normal diet without any additive, no challenge. Control is normal diet with the piglets challenged with E. coli. Treatment groups 3 and 4 are on the Weaning Supplement fed at 50 ppm and 100 ppm.

FIG. 3 depicts that the Weaning Supplement fed at 50 ppm had an improved FCR (that is G:F weight gain to feed intake ratio, though the body weight at 50 ppm was marginally lower than the control. The four treatment groups were considered during the trial. Negative control is normal diet without any additive, no challenge. Control is normal diet with the piglets challenged with E. coli. Treatment group 3 and 4 are the Weaning Supplement fed at 50 ppm and 100 ppm.

FIG. 4 depicts Weaning supplement fed group at 100 ppm had improvement in weight gain FIG. 4a), reduced FCR FIG. 4b) and controlled diarrhea FIG. (4c).

FIG. 5 depicts dose dependent increase in crypt depth in duodenum of weaning supplement composition/product supplemented pigs, 21 days post E. coli infection.

FIGS. 6a and 6b depicts dose dependent increase in villi height and villi surface area in the jejunum region of weaning supplement composition/product supplemented pigs, 5 days post E. coli infection.

FIG. 7 depicts dose dependent increase in villi height to crypt depth ratio in the jejunum region of weaning supplement composition/product supplemented pigs, 5 days post E. coli infection.

FIG. 8 depicts the change in serum TNF-α concentration up on weaning supplement/product supplemented piglets, 4 days post E. coli infection.

FIG. 9 depicts daily change in β-hemolytic colifrom count in feces of piglets challenged with E. coli, both in control and weaning supplement groups.

FIGS. 10a & 10b depicts weekly changes in fecal bacterial load and diarrhea frequency change in weaning product supplemented pigs, compared to control.

FIG. 11 represents the stability of the product in both pellet and mash feed.

### DETAILED DESCRIPTION OF THE INVENTION

The invention will now be described in detail in connection with certain preferred and optional embodiments, so that various aspects thereof may be more fully understood and appreciated.

### Geographical Origin and Source:

Capsicum oleoresin
Geographical Origin: Native to southern North America, the Caribbean, and northern South America.
Source: China

### Clove Oil (Includes Clove Leaf Oil, Clove Bud Oil)

Geographical Origin: Native to Indonesia, harvested primarily in Zanzibar, Indonesia and Madagascar. Also grown in Indi a, Pakistan and Sri Lanka.
Source: Indonesia

### Garlic Oil (Includes Garlic Bud Oil)

Geographical Origin: Native to South Asia, Central Asia and northeastern Iran.
Source: China

### Palm Oil

Geographical Origin: The biggest producers of palm oil are Indonesia, Malaysia, Thailand, and Nigeria
Source: Malaysia/Indonesia

### Sunflower Oil

Geographical Origin: Russia and Ukraine
Source: Russia/Ukraine/Argentina

### Soybean Oil

Geographical Origin: The wild soybean is native to China, Japan, Korea and Russia
Source: China/Brazil/Argentina/USA

### Mustard Oil

Geographical Origin: It is native to tropical regions of North Africa, temperate regions of Europe, and parts of Asia Source: Canada/Nepal/Russia

### Rice Bran

Geographical Origin: It is popular as a cooking oil in East Asia, the Indian subcontinent, and Southeast Asia including India, Nepal, Bangladesh, Indonesia, Japan, Southern
China and Malaysia.
Source: —Bangladesh/Nepal/China

### Rapeseed Oil

Geographical Origin: China, Japan, India, Northern Europe
Source: —Netherlands

### Gum Acacia

Geographical Origin: It is a native to the Sudan region in Africa
Source: —France

Accordingly, the present invention provides a weaning supplement composition that can improve performance by improving intestinal health, function, and structure in animals and human beings. The composition of the present invention can decrease susceptibility to post-weaning diarrhea as well as potential downstream illnesses such as gut and respiratory disease and further improves weight gain of the animals and human beings.

Accordingly in one embodiment, the invention relates to a weaning supplement composition which comprises;
a) Capsicum oleoresin in an amount of 0.1 to 10%;
b) Clove oil in an amount of 0.1 to 20%;
c) Garlic oil in an amount of 0.1 to 20%;
d) Emulsifiers in an amount of 0 to 30%; and
e) Water soluble carrier or fat soluble carrier in an amount of 50 to 99%,

The water-soluble carrier may be selected from the group consisting of gum arabica, Modified starch/PVP, maltodextrin, sucrose, lactose, sodium chloride, maltose, gum acacia, dextrin, cyclodextrin, xanthan gum, guar gum and the like.

In further embodiment, the water-soluble carrier may be selected from the group consisting of either individually or as a blend of above, in any ratio and up to 60 to 90%.

In another embodiment, the invention discloses water-soluble weaning supplement composition which comprises;
a) Capsicum oleoresin in an amount of 0.1 to 10%;
b) Clove oil in an amount of 0.1 to 20%;
c) Garlic oil in an amount of 0.1 to 20%;
d) Emulsifiers in an amount of 0 to 30%; and
e) Water soluble carrier in an amount of 50 to 99%,

The emulsifiers/surfactants/wetting agents/solvents may be selected from the group consisting of synthetic source.

In another embodiment, the emulsifier/surfactant/wetting agents/solvents is selected in an amount of 30-99%, more preferably 60-90%, from the group consisting of polysorbates, span, propylene glycol, glycerol, polyethylene glycol, and the like either alone or combinations thereof.

In further embodiment, the emulsifier/surfactant/wetting agents/solvents may be selected from the group consisting of either individually or as a blend of above, in any ratio and up to 60 to 90%.

In yet another embodiment, the invention relates to a fat encapsulated weaning supplement composition which comprises;
a) Capsicum oleoresin in an amount of 0.1 to 10%;
b) Clove oil in an amount of 0.1 to 20%;
c) Garlic oil in an amount of 0.1 to 20%;
d) Vegetable oil(s) in an amount of 0 to 30%; and
e) Fat soluble carrier in an amount of 50 to 99%,

The composition is encapsulated in fat soluble carrier wherein the fat-soluble carrier(s) is preferably a vegetable fat.

In an embodiment, the vegetable oil is optionally selected in an amount of 0.1-30%, more preferably in an amount of 1%-15%, from the group consisting of Palm, sunflower oil, Soybean, mustard oil, rice bran oil, rapeseed oil either alone or combination thereof.

Accordingly, the vegetable oil may be selected from the blend of any of the above oils in any ratio. However, in one of the preferred embodiments, the vegetable oils used for example, may be selected from Palm, sunflower oil, Soybean, mustard oil, rice bran oil, rapeseed oil, in an amount of 0.1 to 30% preferably in an amount of 0.5 to 20%; more preferably in an amount of 1% to 15% based upon the total weight of the weaning supplement composition.

In an embodiment, the vegetable oil may be present in the weaning supplement composition in an amount of 0.1 to 30%;

In another embodiment, the vegetable fat is selected in an amount of 50-99%, preferably in an amount of 60 to 90%; more preferably in an amount of 70% to 85%, based upon the total weight of the weaning supplement composition from the group consisting of Hydrogenated vegetable oils or oleic and stearic fatty acids. In a further embodiment, the vegetable fat may be selected from the group consisting of either individually or as a blend of Hydrogenated vegetable oils, Glycerol mono stearate, Rapeseed oil in any ratio and upto 80 to 90%.

In one of the preferred embodiment, the hydrogenated vegetable oil may be selected from Palm stearin, rape seed oil, Carnauba wax in an amount of 10 to 99%.

The fat encapsulated weaning supplement composition is free-flowing and homogeneous in nature. These are stable to pelleting conditions and can be mixed with both mash and pellet animal feed in an amount of preferably 50 to 1000 ppm by weight of the animal feed.

The use of water soluble or fat-soluble carrier gives the flexibility of the weaning supplement composition to be provided to the animals as liquid diet for example, along with milk replacer or as a solid diet for example, as a feed supplement, or direct fed by top dressing the feed.

In an embodiment, the capsicum oleoresin, standardized to 5% to 60% total capsaicinoids, may be present in the weaning supplement composition in an amount of 0.1 to 10%; preferably in an amount of 0.5 to 5%; more preferably in an amount of 1% to 8% based upon the total weight of the weaning supplement composition.

In an embodiment, the clove oil may be selected from Clove leaf oil, clove bud oil etc. including natural, nature identical and synthetic containing eugenol content 5% to 30%. Accordingly, the clove oil may be present in the weaning supplement composition in an amount of 0.1 to 20%; preferably in an amount of 0.5 to 10%; more preferably in an amount of 1% to 15% based upon the total weight of the weaning supplement composition.

In an embodiment, the garlic oil may be selected from Garlic bud oil of natural, nature identical and synthetic containing DADS (diallyl disulphide) 5% to 50%. Accordingly, garlic oil may be present in the weaning supplement composition in an amount of 0.1 to 20%; preferably in an amount of 0.5 to 10%; more preferably in an amount of 1% to 15% and based upon the total weight of the weaning supplement composition.

In an embodiment the weaning supplement composition may be fed to the animal either directly or by mixing with the feed/water/milk replacer, at an inclusion rate from about 50 ppm to 10000 ppm; preferably in an amount of 100 ppm to 10000 ppm, more preferably in an amount of 50 ppm to 1000 ppm, based upon the total weight of the feed, water or milk replacer as the case may be, taken as 100% by weight.

The weaning supplement composition includes free-flowing, homogeneous particles and therefore can be further formulated into dust free granules, pellets or capsules that can be mixed with animal feed in an amount of preferably 100 to 1000 ppm by weight of the animal feed or can be given by weight of the milk replacement.

In an embodiment the weaning supplement composition may be fed to the cattle dose as 50 to 1000 ppm in feed or 50 mg to 1000 mg per head per day.

In another embodiment the weaning supplement composition may be administered to human beings as Human dose of 10 mg to 3000 mg per day/individual.

In yet another embodiment, the invention relates to a process for preparation of weaning supplement composition which process comprises the steps of;
a) melting the fat-soluble carrier selected from vegetable fat at a temperature range of 40 to 80° C. under continuous stirring;
b) adding Capsicum oleoresin followed by Garlic oil, Clove oil under continuous stirring till to get homogenous mixture;
c) Spray encapsulation
d) cooling the mixture of step b) followed by passing the mixture through suitable sieves to obtain fat encapsulated weaning supplement composition.

In a further embodiment, the process step for preparation of water-soluble weaning supplement composition which process comprises the steps of;
a) homogenizing the water-soluble carriers selected from emulsifiers in water/suitable solvents followed by addition Capsicum oleoresin, garlic oil, clove oil under stirring to obtain homogenized blend 1;
b) dissolving carriers/encapsulating agents such as maltodextrin in water/suitable solvents under stirring to obtain homogenized blend 2;
c) adsorbing or encapsulating the Blend 1 on to blend 2, drying, followed by adding anticaking agent and blending to obtain the free-flowing granules

In yet another embodiment, the invention provides a Spray encapsulation process for preparation of weaning supplement composition which process comprises the steps of;
a) preparing the blend of all the oleoresin, surfactant and herbal oils, well at 40 to 70° C. to obtain-Blend 1;
b) Dissolving the maltodextrin in water to obtain-Blend 2;
c) Adding Blend 1 to Blend 2 under stirring at 50° C. for 1 Hr;
d) Setting the Nozzle as per product size requirement, passing steam by maintaining the temperatures to collect the encapsulated powder in liners;
e) adding anticaking agent, blending and sieving to get the free-flowing powder.

The inventive step of the weaning supplement composition lies in the judicial combination of capsicum oleoresin, garlic oil, clove oil along with vegetable oil in a specific ratio which works synergistically and thus improves the intestinal health, function, and structure of the animals. The animals may be selected from not only piglets but also calves, receiving cattle, human beings and other young live stock.

The weaning supplement composition may be administered at a minimum dose of 100 ppm per ton of the feed or 50 ppm to 1000 ppm per 1000 L of milk replacer, as the case may be.

In yet another embodiment, the weaning supplement composition can be supplemented in creep feed and nursery diets to decrease the negative impact of weaning on enteric health. Decreased clinical disease and improved efficiency of growth during an enteric challenge are consistent with the mechanism of decreased inflammation, improved intestinal structure, and increased digestive function in animals supplemented with the weaning supplement composition of the present invention.

Accordingly, in yet another embodiment, the invention provides method of improving gut barrier function, decreasing gut inflammation and increasing nutrient absorption in an animal, which method comprises, supplementing the animal in need thereof, a weaning supplement composition comprising capsicum oleoresin, garlic oil, clove oil and vegetable oil encapsulated in vegetable matrix, provided according to the present invention in an amount of 50 ppm to 1000 ppm by weight of the animal feed or milk replacer.

In a further embodiment, the invention provides weaning supplement compositions comprising capsicum oleoresin, garlic oil, clove oil and vegetable oil encapsulated in vegetable matrix in an amount of 50 ppm to 1000 ppm by weight of the animal feed, for use in improving gut barrier function, decreasing gut inflammation and increasing nutrient absorption in an animal.

The following examples are for illustration purpose of the invention only and are not intended to limit the scope of the present invention in any manner.

### EXAMPLES

The following examples are given by way of illustration and therefore should not be construed to limit the scope of the invention.

### Example 1

### Weaning Supplement Composition:

TABLE 1

| Sr. No | Ingredient | Quantity/range |
|---|---|---|
| 1 | Capsicum Oleoresin | 0.1-10% |
| 2 | Clove oil | 0.1-20% |
| 3 | Garlic oil | 0.1-20% |
| 4 | Vegetable oil | 3% |
| 5 | Vegetable fat (Hydrogenated vegetable | 50-99% |
| | oil) | |
| 6 | Calcium stearate/Silicon dioxide Optional | 0-2% |

### Example 2

TABLE 2

| Sr. No | Ingredient | Quantity/range |
|---|---|---|
| 1 | Capsicum Oleoresin | 1% |
| 2 | Clove oil | 10% |
| 3 | Garlic oil | 6% |
| 4 | Sunflower oil | 3% |
| 5 | Hydrogenated palm oil | 80% |
| 6 | Calcium stearate/Silicon dioxide Optional | 0-2% |

### Example 3

TABLE 3

| Sr. No | Ingredient | Quantity/range |
|---|---|---|
| 1 | Capsicum Oleoresin | 5% |
| 2 | Clove oil | 5% |
| 3 | Garlic oil | 10% |
| 4 | Palm oil | 9% |
| 5 | Hydrogenated Rapeseed oil | 70% |
| 6 | Calcium stearate/Silicon dioxide Optional | 0-2% |

### Example 4

TABLE 4

| Sr. No | Ingredient | Quantity/range |
|---|---|---|
| 1 | Capsicum Oleoresin | 10% |
| 2 | Clove oil | 20% |
| 3 | Garlic oil | 20% |
| 4 | Rice bran oil | 7% |
| 5 | Glyceryl monostearate | 43% |
| 6 | Calcium stearate/Silicon dioxide Optional | 0-2% |

### Example 5

TABLE 5

| Sr. No | Ingredient | Quantity/range |
|---|---|---|
| 1 | Capsicum Oleoresin | 10% |
| 2 | Clove oil | 20% |
| 3 | Garlic oil | 20% |
| 4 | Sunflower oil | 5% |
| 5 | Hydrogenated rapeseed oil | 43% |
| 6 | Calcium stearate/Silicon dioxide Optional | 0-2% |

### Example 6

TABLE 6

| Sr. No | Ingredient | Quantity/range |
|---|---|---|
| 1 | Capsicum Oleoresin | 1% |
| 2 | Clove oil | 10% |
| 3 | Garlic oil | 6% |
| 4 | Vegetable fat (Hydrogenated vegetable oil | 83% |
| | from Rapeseed/Palm) | |
| 5 | Calcium stearate/Silicon dioxide Optional | 0-2% |

### Example 7

TABLE 7

| Sr. No | Ingredient | Quantity/range |
|---|---|---|
| 1 | Capsicum Oleoresin | 5% |
| 2 | Clove oil | 5% |
| 3 | Garlic oil | 10% |
| 4 | Vegetable fat (Hydrogenated vegetable oil | 80% |
| | from rape seed/palm) | |
| 5 | Calcium stearate/Silicon dioxide Optional | 0-2% |

### Example 8

TABLE 8

| Sr. No | Ingredient | Quantity/range |
|---|---|---|
| 1 | Capsicum Oleoresin | 10% |
| 2 | Clove oil | 20% |
| 3 | Garlic oil | 20% |
| 4 | Vegetable fat (Hydrogenated vegetable oil. | 50% |
| | Glyceryl monostearate) | |
| 5 | Calcium stearate/Silicon dioxide Optional | 0-2% |

### Example 9

TABLE 9

| Sr. No | Ingredient | Quantity/range |
|---|---|---|
| 1 | Capsicum Oleoresin | 0.25% |
| 2 | Clove oil | 4% |
| 3 | Garlic oil | 2% |
| 4 | Vegetable oil | <2% |
| 5 | Vegetable fat (Hydrogenated vegetable oil | 93% |
| | from palm/rapeseed) | |
| 6 | Calcium stearate/Silicon dioxide Optional | 0-2% |

### Example 10

TABLE 10

| Sr. No | Ingredient | Quantity/range |
|---|---|---|
| 1 | Capsicum Oleoresin | 2.5% |
| 2 | Clove oil | 8% |
| 3 | Garlic oil | 5% |
| 4 | Vegetable oil | <2% |
| 5 | Vegetable fat (Hydrogenated vegetable oil | 84% |
| | from palm/rapeseed) | |
| 6 | Calcium stearate/Silicon dioxide Optional | 0-2% |

### Manufacturing Procedure:

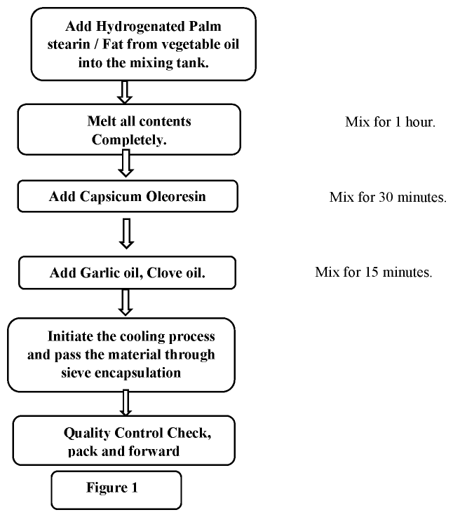

1. Transferred the Batch quantity of Vegetable Oil/Vegetable Fat (Hydrogenated vegetable oil selected from Palm oil, Sunflower oil, Soybean oil, Mustard oil, Rice bran, Rapeseed oil) into melting tank.
2. Maintained the melting tank temperature 75° to 80° C. under continuous stirring for 1 hour.
3. Reduced the melting tank temperature to 70° C. Added batch quantity of Capsicum oleoresin, mixed for 30 minutes. Added Garlic oil, Clove oil and mixed for 15 minutes.
4. Spray congealing is done and passed the material through suitable sieves.
5. Collected the finished product, and packed after the quality check is passed. The process is depicted as a flow chart in FIG. 1.

### Weaning Supplement Composition

### Example 11

TABLE 11

| Sr. No | Ingredient | Quantity |
|---|---|---|
| 1 | Capsicum Oleoresin | 0.1 to 10% |
| 2 | Clove oil | 0.1 to 20% |
| 3 | Garlic oil | 0.1 to 20% |
| 4 | Vegetable oil (Sunflower oil/rapeseed oil/ | 0.1 to 30% |
| | sunflower oil, Soybean, mustard oil, rice | |
| | bran oil,) | |
| 5 | Vegetable fat (hydrogenated vegetable fat | 50 to 99-% |
| | from Palm) | |
| 6 | Silicon dioxide | 0 to 2% |

### Example 12

TABLE 12

| Sr. | | |
|---|---|---|
| No. | Ingredient Name | % Range |
| 1 | Capsicum Oleoresin | 1% |
| 2 | Garlic Oil | 6% |
| 3 | Clove leaf Oil | 10% |
| 4 | Gum arabica | 4% |
| 5 | Water soluble carrier | 79% |
| | Modified starch/PVP/ | |
| | starch derivative | |
| 6 | Silicon dioxide | 0 to 2% |

### Example 13

TABLE 13

| Sr. | | |
|---|---|---|
| No. | Ingredient Name | % Range |
| 1 | Capsicum Oleoresin | 1% |
| 2 | Garlic Oil | 6% |
| 3 | Clove leaf Oil | 10% |
| 4 | Gum arabica | 50% |
| | Polysorbate/Emulsifier | 5 |
| 5 | Water soluble carrier | 28% |
| | Modified starch/PVP/ | |
| | starch derivative | |
| 6 | Silicon dioxide | 0 to 2% |

### Example 14

TABLE 14

| Sr. | | |
|---|---|---|
| No. | Ingredient Name | % Range |
| 1 | Capsicum Oleoresin | 10% |
| 2 | Garlic Oil | 20% |
| 3 | Clove leaf Oil | 20% |
| 4 | Gum arabica/ | 25% |
| | Polysorbate/Emulsifier/ | 5 |
| 5 | Water soluble carrier | 20% |
| | Modified starch/PVP/ | |
| | starch derivative | |
| 6 | Silicon dioxide | 0 to 2% |

### Example 15

TABLE 15

| Sr. No. | Ingredient Name | % Range |
|---|---|---|
| 1 | Capsicum Oleoresin | 10% |
| 2 | Garlic Oil | 20% |
| 3 | Clove leaf Oil | 20% |
| 4 | Gum arabica/ | 25% |
| 5 | Water soluble carrier Modified | 25% |
| | starch/PVP/starch derivative | |
| 6 | Silicon dioxide | 0 to 2% |

### Example 16

TABLE 16

| Sr. No. | Ingredient Name | % Range |
|---|---|---|
| 1 | Capsicum Oleoresin | 5% |
| 2 | Garlic Oil | 10% |
| 3 | Clove leaf Oil | 5% |
| 4 | Polysorbate/Emulsifier/ | 5% |
| 5 | Water soluble carrier Modified | 75% |
| | starch/PVP/starch derivative | |
| 6 | Silicon dioxide | 0 to 2% |

### Example 17

TABLE 17

| Sr. No. | Ingredient Name | % Range |
|---|---|---|
| 1 | Capsicum Oleoresin | 5% |
| 2 | Garlic Oil | 10% |
| 3 | Clove leaf Oil | 5% |
| 4 | Gum arabica/ | 0% |
| 5 | Water soluble carrier Modified | 80% |
| | starch/PVP/starch derivative | |
| 6 | Silicon dioxide | 0 to 2% |

### Manufacturing Procedure of Spray Encapsulation:

### Blend-1

Homogenized Gum Arabica/Polysorbate with water.

Added all oils+oleoresin, polysorbate and homogenize for 1 hr to obtain Blend 1

### Blend-2

Dissolved Water soluble carrier in water and homogenized to obtain blend 2

### Final Process

Blend 1 is adsorbed on to blend 2, spray dried, followed by addition of anticaking agent and blended to obtain the free-flowing granules

### Example 18

TABLE 18

| Sr. No. | Ingredient Name | % Range |
|---|---|---|
| 1 | Capsicum Oleoresin | 0.1 to 10% |
| 2 | Garlic Oil | 0.1 to 20% |
| 3 | Clove leaf Oil | 0.1 to 20% |
| 4 | Polysorbate | 0.1 to 30% |
| 5 | Gum Arabica/Modified | 10 to 99% |
| | starch/starch derivative | |
| 6 | Silicon dioxide | 0 to 2% |

### Example 19

TABLE 19

| Sr. No. | Ingredient Name | % Range |
|---|---|---|
| 1 | Capsicum Oleoresin | 1% |
| 2 | Garlic Oil | 6% |
| 3 | Clove leaf Oil | 10% |
| 4 | Polysorbate | 83% |
| 5 | Propylene glycol/ | 0% |
| | Glycerol/PEG400 | |

### Example 20

TABLE 20

| Sr. No. | Ingredient Name | % Range |
|---|---|---|
| 1 | Capsicum Oleoresin | 1% |
| 2 | Garlic Oil | 6% |
| 3 | Clove leaf Oil | 10% |
| 4 | Polysorbate | 30% |
| 5 | Propylene glycol/ | 53% |
| | Glycerol/PEG400 | |

### Example 21

TABLE 21

| Sr. No. | Ingredient Name | % Range |
|---|---|---|
| 1 | Capsicum Oleoresin | 10% |
| 2 | Garlic Oil | 20% |
| 3 | Clove leaf Oil | 20% |
| 4 | Polysorbate | 5% |
| 5 | Propylene glycol/ | 45% |
| | Glycerol/PEG400 | |

### Example 22

TABLE 22

| Sr. No. | Ingredient Name | % Range |
|---|---|---|
| 1 | Capsicum Oleoresin | 5% |
| 2 | Garlic Oil | 10% |
| 3 | Clove leaf Oil | 5% |
| 4 | Polysorbate | 5% |
| 5 | Propylene glycol/ | 75% |
| | Glycerol/PEG400 | |

### Example 23

TABLE 23

| Sr. No. | Ingredient Name | % Range |
|---|---|---|
| 1 | Capsicum Oleoresin | 10% |
| 2 | Garlic Oil | 20% |
| 3 | Clove leaf Oil | 20% |
| 4 | Polysorbate | 30% |
| 5 | Propylene glycol/ | 20% |
| | Glycerol/PEG400 | |

### Example 24

TABLE 24

| Sr. No. | Ingredient Name | % Range |
|---|---|---|
| 1 | Capsicum Oleoresin | 5% |
| 2 | Garlic Oil | 10% |
| 3 | Clove leaf Oil | 5% |
| 4 | Polysorbate | 30% |
| 5 | Propylene glycol/ | 50% |
| | Glycerol/PEG400 | |

### Manufacturing Procedure of Liquid:

### Procedure

1. Taken the given quantity of capsicum oleoresin, polysorbate and oils and blend well at a temperature range of 60-70° C. to obtain Blend 1.
2. Taken given quantity of solvent like Propylene glycol/Glycerol/PEG 400,/Polysorbate blended well to obtain blend 2.
3. Added Blend 1 to Blend 2 under stirring for 1 Hr to obtain homogenized mixture.
4. Collected the liquid and stored until quality check, then dispatched

### Example 25

TABLE 25

| Sr. No. | Ingredient Name | % Range |
|---|---|---|
| 1 | Capsicum Oleoresin | 5% |
| 2 | Garlic Oil | 10% |
| 3 | Clove leaf Oil | 5% |
| 4 | Polysorbate | 80% |

### Example 26

### to Study the Efficacy of the Weaning Supplement Composition in Decreasing the Negative Impact of Post-Weaning Diarrhea

The efficacy of the weaning supplement composition in decreasing the negative impact of post-weaning diarrhea was conducted in nursery piglets weighing average 7 Kgs, taken 8 number of animals in each of the four groups,
1. Negative control is normal diet without any additive, no challenge.
2. Control is normal diet with the animals challenged with E. coli.

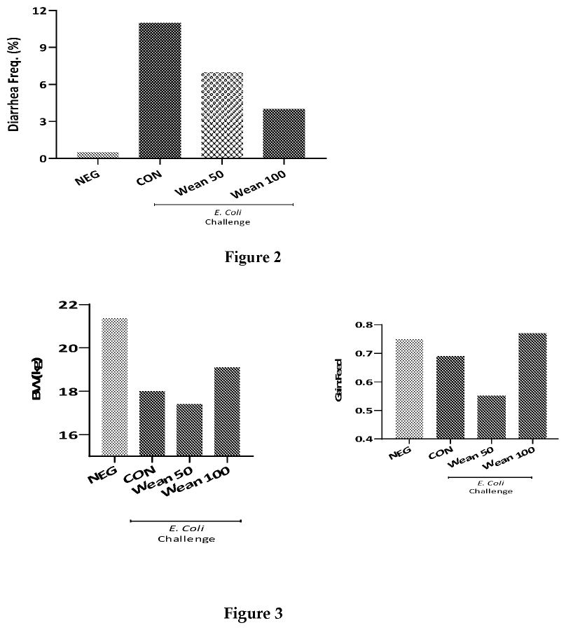

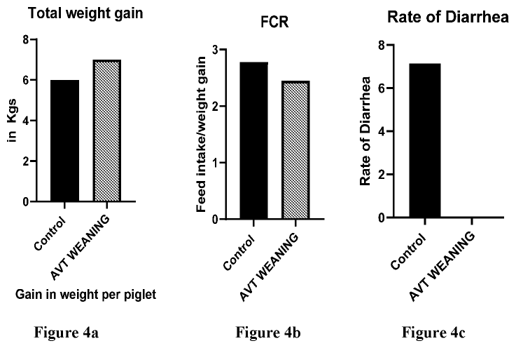

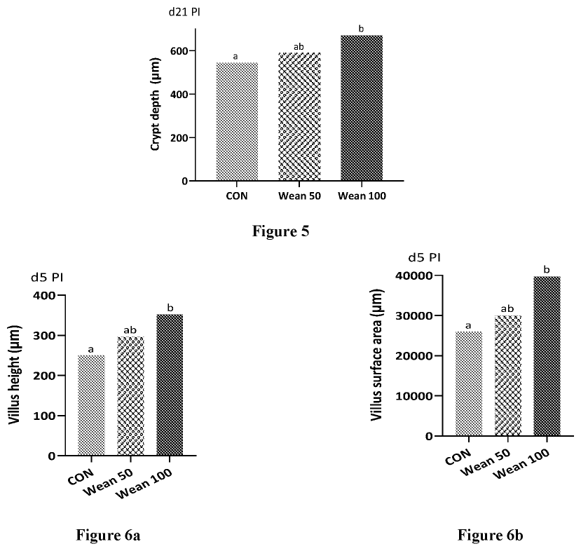

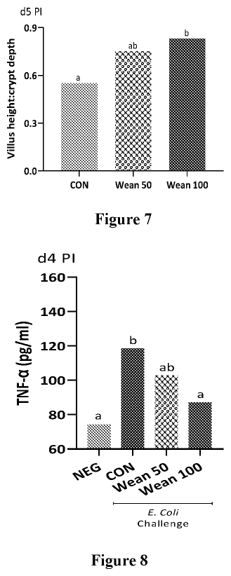

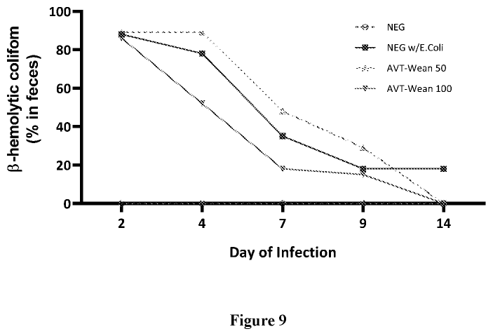

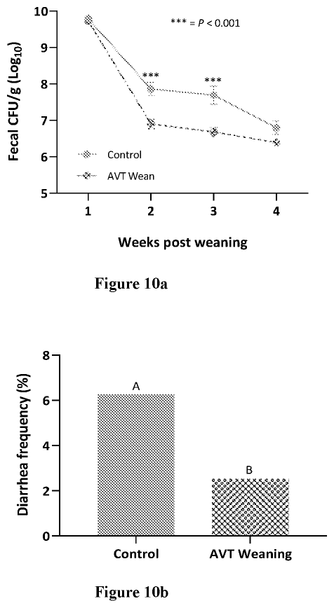

Treatment group 3 and 4 are the Weaning Supplement at 50 ppm and 100 ppm animals challenged with E. coli. which were supplemented with weaning supplement composition of example 1 with vegetable fat—spray encapsulation. Performance was monitored for 21 days to measure the disease in severity and recovery. The FIG. 2 is discussed in below table 1.

TABLE 26

| | | | E. coli | E. coli |
|---|---|---|---|---|
| | | Control with | challenge with | challenge with |
| Treatment | Negative | E. coli | WEANING @ | WEANING @ |
| group | control | challenge | 50 ppm | 100 ppm |
| Diarrhea | 0.5 | 11 | 7 | 4 |
| frequency | | | | |
| in % | | | | |

The Weaning Supplement fed at 50 ppm and 100 ppm had a reduced diarrheal frequency with challenged E. coli, when compared to Controlled challenged with E. coli having a high diarrheal frequency.

As shown in the FIG. 2 and the above table 1, frequency of diarrhea during peak disease was markedly reduced in the piglets supplemented with 100 ppm of weaning supplement composition.

This pattern held out through d 21 post infection, with decreased fecal scores during disease and overall, in the WEAN 100 pigs (piglets supplemented with the 100 ppm of the composition). Performance was also increased in that treatment group, with improved gain-to-feed and increased body weight on d21 post infection, as shown in FIG. 3 and table below.

As shown in below table 27, the Weaning Supplement fed at 50 ppm had an improved FCR (that is G:F weight gain to feed intake ratio, though the body weight at 50 ppm was marginally lower than the control.

TABLE 27

| | | | E. coli | E. coli |
|---|---|---|---|---|
| | | Control with | challenge with | challenge with |
| Treatment | Negative | E. coli | WEANING @ | WEANING @ |
| group | control | challenge | 50 ppm | 100 ppm |
| Gain: Feed | 0.75 | 0.69 | 0.55 | 0.77 |
| Body weigh | 21.38 | 18 | 17.4 | 19.1 |
| in Kg | | | | |

Intestinal sections of control and weaning supplement composition/product supplemented animals were collected in E. coli challenged pigs 5 days and 21 days post infection and studied for intestinal structural changes. Study affirmed that Weaning supplement composition/product supplementation improved the intestinal integrity and structure as evident by dose dependent increase in crypt depth, villi height, villi surface area and villi to crypt depth ratio. (FIGS. 6 (6a & 6b) to 7)

TNF-α an important a major regulator of inflammatory responses, increased in unsupplemented control group piglets challenged with E. coli. This might trigger cascade of inflammatory reactions and hence can affect performance of the animals. Weaning supplement/product supplemented piglets had shown dose dependent decrease in serum TNF-α levels, indicating ani-inflammatory, preventing gut damage and disease resistance properties of the supplement (Figure number 8).

β-hemolytic colifroms are pathogenic in nature and it is important to control its count in the gut to obtain optimal growth and performance. In E. coli challenged piglets, β-hemolytic colifroms count was reduced upon 100 ppm of weaning supplement treatment (Figure number 9). This affirms that weaning product positively alter the gut microbiome and offers resistance to pathogen.

### CONCLUSION

As is evident from the FIGS. 2 to 3, the weaning supplement composition of the present invention improves intestinal health, function, and structure, thereby decreasing susceptibility to post-weaning diarrhea as well as potential downstream illnesses such as respiratory disease.

The weaning supplement composition further provides improved gut barrier function, decreased gut inflammation and increased nutrient absorption and thereby leading to decreased diarrhea, increased daily gains and improved feed efficiency. The supplementation elicits marked changes at the level of the intestine that are weak points during the weaning transition. These responses then translate into improved performance.

### Example 27

### to Study the Effect of Stress Caused During Weaning

Piglets after weaning were considered for the study. 16 piglets were sorted into 02 groups with 8 piglets per group. Piglets in group 1 were given normal feed, group 2 was supplemented with AVT weaning at 100 ppm in feed . . .

TABLE 28

| | Control | AVT WEANING @100 ppm |
|---|---|---|
| Total weight gain (kg) | 5.990 | 7.000 |
| Average daily feed intake (g) | 595.100 | 611.800 |
| FCR | 2.782 | 2.447 |

Weaning stress on gut and intestinal health has a major impact on the Performance of the Piglets. Therefore, it is necessary to reduce physiological changes in the intestine caused by stress.

In the above study the effect of stress caused during weaning has been studied. The Weaning supplemented group supplemented by the composition of the present invention showed a significance effect in the intestinal effect and thereby the overall performance of the piglets.

### Example 28

### to Study the Effect of Stress Caused During Weaning

Piglets after weaning were considered for the study and were sorted into 02 groups with 8 piglets per group. Piglets in group 1 were given normal feed, group 2 was supplemented with AVT weaning at 100 ppm in feed.

TABLE 29
Summary of overall performance results of piglets in control and
weaning supplement composition/product supplemented groups

| | Control | AVT Wean | SEM | P-value |
|---|---|---|---|---|
| Weaning weight, g | 685 | 695 | ±12 | 0.53 |
| Body weight d 28, kg | 14.9 | 18.2 | ±0.4 | 0.001 |
| Total gain d 1-28, kg | 8.05 | 11.25 | ±40 | 0.001 |
| ADFI d 1-28, g | 530 | 528 | ±14 | 0.90 |
| ADG d 1-28, g | 288 | 402 | ±70 | 0.001 |
| Feed: Gain d 1-28 | 1.84 | 1.31 | ±0.10 | 0.001 |

Table 29 above consolidates the summary of overall performance results of piglets in control and weaning supplement composition/product supplemented groups.

Overall summary consolidated in table 4 affirms that weaning supplement composition/product supplementation improves the performance of piglets in spite of the weaning stress, interms of improvement in body weight, total gain, average daily gain and feed to gain ratio.

In week 2 and 3 of weaning stress, significant difference in fecal bacterial load was observed in weaning supplement composition/product supplemented groups (Figure Number 10a & 10b). This result and the diarrhea frequency data are considered together it is obvious that the supplementation has reduced the pathogen shedding and spread and hence less frequency of diarrhea.

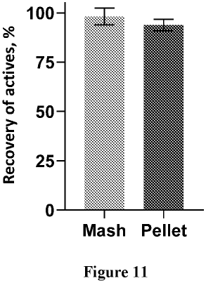

FIG. 11 represents the stability of the product in both pellet and mash feed.

Total capsaicinoids a marker compound in the weaning supplement composition/product was measured in mash and pellet feed to ascertain the stability of this composition in pelleting condition. After blending weighed quantity of composition in mash feed, part of the feed was subjected to palletization, where feed was exposed to 85° C. for 10 minutes and then the formed pellets and mash feed both were subjected to total curcuminoid analysis using standardized HPLC method. More than 94% of actives were recovered in pellet feed when compared to mash feed, indicating stability of the composition/product to pelleting condition.

### Advantages of the Invention

1. The present invention to provides a weaning supplement composition that can reduce diarrhea incidence and also improve performance and general health of weaning animals and human beings.
2. The weaning supplement composition is pellet-stable, and is 100% dust free.
3. In addition to its ability in decreasing susceptibility to post-weaning diarrhea, this weaning supplement is expected to have potential to decrease downstream illnesses such as respiratory disease in weaning animals.
4. The weaning supplement composition may improve intestinal health, function, and structure of the host, to accomplish above said benefits.
5. The composition of the present invention shall be supplemented directly (top fed) or in directly by mixing with the feed, water and/or milk replacers, during weaning and nursery stages.
6. The Weaning supplement will have anti-inflammatory effect
7. It can be supplemented with other pre-biotics. Post-biotics, anti biotics, organic acids, or any other gut health formulation, for enhanced efficacy
8. This Weaning supplement also has a positive impact on gut microbiome.

### REFERENCE

1. Enríquez D., M. J. Hötzel & R. Ungerfeld. 2011. Minimising the stress of weaning of beef calves: a review. Acta Veterinaria Scandinavica. Vol. 53.
2. Rhouma M., J. M. Fairbrother, F. Beaudry & A. Letellier. 2017. Post weaning diarrhea in pigs: risk factors and non-colistin-based control strategies. Acta Veterinaria Scandinavica. Vol. 59.
3. Liu Y., M. Song, T. M. Che, J. A. S. Almeida, J. J. Lee, D. Bravo, C. W. Maddox, J. E. Pettigrew. 2013. Dietary plant extracts alleviate diarrhea and alter immune responses of weaned pigs experimentally infected with a pathogenic Escherichia coli. Journal of Animal Science. Vol. 91. P. 5294-5306.

1. A weaning supplement composition encapsulated in a fat soluble carrier or a water-soluble carrier for an animal comprising:
a) capsicum oleoresin in an amount of 0.1 to 10%;
b) clove oil in an amount of 0.1 to 20%;
c) garlic oil in an amount of 0.1 to 20%;
d) emulsifiers or vegetable oil(s) in an amount of 0 to 30%; and
e) a fat soluble carrier or a water-soluble carrier in an amount of 50 to 99%,

2. The composition as claimed in claim 1, wherein the oleoresin is present in an amount of 0.5 to 5% of the total weight of the weaning supplement composition.

3. The composition as claimed in claim 1, wherein the composition further comprises vegetable oil.

4. The composition as claimed in claim 1, wherein the water-soluble carrier comprises gum arabica, maltodextrin, sucrose, lactose, sodium chloride, maltose, gum acacia, dextrin, cyclodextrin, xanthan gum, guar gum, or a mixture thereof in an amount of 60% to 90% of the total weight of the weaning supplement composition.

5. The composition as claimed in claim 1, wherein the fat-soluble carrier is a vegetable fat comprising a hydrogenated vegetable oil or an oleic and a stearic fatty acid, rapeseed oil, almond oil, canola oil, soy oil, cotton seed oil, castor oil, glycerol mono stearate, beeswax, carnauba wax, or a mixture thereof in an amount of 50 to 99% of the total weight of the weaning supplement composition

6. The composition as claimed in claim 1, wherein the clove oil comprises clove leaf oil or clove bud oil in an amount of 0.1 to 20% of the total weight of the weaning supplement composition.

7. The composition as claimed in claim 1, wherein the garlic oil comprises garlic bud oil in an amount of 0.1 to 20% of the total weight of the weaning supplement composition.

8. The composition as claimed in claim 1, wherein the capsicum oleoresin is standardized to 5% to 60% total capsaicinoids.

9. The composition as claimed in claim 6, wherein a eugenol content in the clove oil is 5% to 50%.

10. The composition as claimed in claim 7, wherein diallyl disulphide in the garlic oil is 5% to 50%.

11. The composition as claimed in claim 3, wherein the vegetable oil comprises palm, soybean, sunflower, rapeseed, cotton, coconut, olive, rice bran, mustard, sesame, or a mixture thereof in an amount of 0.1 to 30% of the total weight of the weaning supplement composition.

12. The composition as claimed in claim 1, wherein the composition is mixed with both mash and pellet animal feed or water or milk replacer or given directly in an amount of 50 ppm to 10000 ppm by weight of the mash and pellet animal feed or water or milk replacer taken as 100% by weight.

13. The composition as claimed in claim 1, wherein the composition is in the form of a granule, powder, a bead, a pellet, a tablet, a capsule, or a liquid.

14. A process for preparing a weaning supplement composition comprising the steps of:
a) melting a fat-soluble carrier, vegetable fat at a temperature range of 40 to 80° C. under continuous stirring;
b) adding capsicum oleoresin followed by garlic oil and clove oil under continuous stirring to get a homogenous mixture;
c) spray encapsulating;
d) cooling the homogenous mixture of step b) followed by passing the homogenous mixture through a suitable sieves to obtain a fat encapsulated weaning supplement composition.

15. A process for preparation of a water-soluble weaning supplement composition wherein a spray encapsulation process step comprises the steps of:
a) homogenizing a water-soluble carrier comprising an emulsifier in a water/suitable solvents followed by addition of a garlic oil and a clove oil under stirring to obtain a homogenized blend 1;
b) dissolving a carrier/encapsulating agent in a water/suitable solvent under stirring to obtain a homogenized blend 2;
c) adsorbing or encapsulating the homogenized blend 1 on to homogenized blend 2, drying, followed by adding an anticaking agent and blending to obtain a free-flowing granule.

16. A process for preparation of a water-soluble weaning supplement composition wherein the spray encapsulation process step comprises the steps of:
a) preparing a blend of oleoresin, a surfactant, and an herbal oil, at 40 to 700° C. to obtain a blend 1;
b) dissolving the maltodextrin in water to obtain a blend 2;
c) adding the blend 1 to the blend 2 under stirring at 50° C. for 1 hr;
d) setting a nozzle as per product size requirement, passing steam by maintaining the temperatures to collect an encapsulated powder in liners;
e) adding an anticaking agent, blending, and sieving to get a free-flowing powder.

17. (canceled)

18. (canceled)

19. The composition as claimed in claim 1, wherein the composition is supplemented directly (top fed) or in directly by mixing with feed, water, and/or a milk replacer, during weaning and nursery stages.

20. (canceled)

21. (canceled)

22. (canceled)

23. (canceled)

24. (canceled)

25. (canceled)

26. (canceled)

27. (canceled)

28. The composition as claimed in claim 1, further comprising a supplement comprising an organic acid, a probiotic, a prebiotic, a postbiotic, an other botanical supplement, and/or an antibiotic.

29. A method for reducing incidence of diarrhea and improving performance and general health of a weaning animals or a human being, the method comprising administering the weaning supplement composition as claimed in claim 1 to the weaning animal or the human being.

30. A method for decreasing downstream illnesses in a weaning animal or a human being, the method comprising administering the weaning supplement composition as claimed in claim 1 to the weaning animal or the human being.
